(19) United States
(12) Patent Application Publication
(10) Pub. No.: US 2021/0333233 A1
(43) Pub. Date: Oct. 28, 2021

(54) SEMICONDUCTOR DEVICE, BIOLOGICAL SIGNAL SENSOR AND BIOLOGICAL SIGNAL SENSOR SYSTEM

(71) Applicant: RENESAS ELECTRONICS CORPORATION, Tokyo (JP)

(72) Inventor: Yasuhiro SHIRAI, Tokyo (JP)

(73) Assignee: RENESAS ELECTRONICS CORPORATION, Tokyo (JP)

(21) Appl. No.: 16/858,320

(22) Filed: Apr. 24, 2020

(51) Int. Cl. G01N 27/414

(52) U.S. Cl. CPC G01N 27/4145; A61B 5/0402

(57) ABSTRACT

A semiconductor device includes a first terminal receiving a first signal, a second terminal receiving a second signal, a noise extraction analysis unit extracting a signal of a specific frequency component from the first and the second signal, a feedback unit generating a feedback signal based on a magnitude of the signal of the specific frequency component to cancel the signal of the specific frequency component superimposed on the first and the second signal, and third terminal outputting to the feedback signal to outside.

### BACKGROUND

The present invention relates to a semiconductor device, a biological signal sensor, and a biological signal sensor system for sensing biological signals by using signal detection electrodes.

A biological signal sensor, such as an electrocardiograph or an electromyograph, detects biological signals using a plurality of signal detection electrodes attached to a human body, amplifies the biological signals using a differential amplifier, and displays or records the amplified biological signals. It is known that common mode noise, such as hum noise or the like caused by a commercial power source, is superimposed on the biological signals obtained from the signal detection electrode.

Patent Document 1 discloses a technique for reducing common mode noise, such as hum noise, by compensating for imbalances in impedances at signal detection electrodes attached on the human body. Non Patent Document 1 discloses a driven-right-leg circuit using the Right Leg Drive method. In Right Leg Drive technique, the inverted amplified signal of the electrocardiographic signal on which the common mode noise is superimposed is fed back to the right foot as a feedback signal.

There are disclosed techniques listed below.
[Patent Document 1] Japanese Unexamined Patent Application Publication 2018-094412
[Non Patent Document 1] “Driven-Right-Leg Circuit Design”, BRUCE B. WINTER and JOHN G. WEBSTER, IEEE TRANSACTIONS ON BIOMEDICAL ENGINEERING, VOL. BME-30, NO. 1, JANUARY 1983

### SUMMARY

The common mode noise is reduced from the biological signal by inverting and amplifying the intermediate potential signal of the two biological signals to be differentially amplified and feeding back the inverted amplified signal to the human body according to Right Leg Drive method. Conventionally, in order to reduce the common mode noise such as hum noise, for example, a biological signal sensor is installed in an environment in which the influence of hum noise does not vary as much as possible to acquire biological signals. However, it is assumed that the wearable biological signal sensors use in a variety of environments. Since the intensity and the frequency component of the hum noise vary depending on the environment, the hum noise superimposed on the biological signal is not reduced depending on the measurement environment, and the target biological signals cannot be displayed.

Other objects and novel features will become apparent from the description of this specification and the accompanying drawings.

According to one embodiment, the semiconductor device comprises a first terminal receiving a first signal, a second terminal receiving a second signal, a noise extraction analysis unit coupled to the first terminal and the second terminal to extract a signal of a specific frequency component from the first signal and the second signal, a feedback unit generating a feedback signal based on a magnitude of the signal of the specific frequency component to cancel the signal of the specific frequency component superimposed on the first and the second signal, and a third terminal outputting the feedback signal to outside.

According to another embodiment, the biological signal sensor includes a first terminal for receiving a first biological signal detected by a first signal detection electrode attached to a human body, a second terminal for receiving a second biological signal detected by a second signal detection electrode attached to the human body, a differential amplifier for differentially amplifying the first biological signal and the second biological signal, a noise extraction analysis unit for extracting a signal of a specific frequency component from an intermediate potential signal of the first biological signal and the second biological signal, a feedback unit for generating a feedback signal for canceling the signal of the specific frequency component superimposed on the first biological signal and the second biological signal according to the magnitude of the signal of the specific frequency component, and a third terminal for outputting a feedback signal to a feedback electrode attached to the human body.

According to one embodiment, the hum noise superimposed on the biological signal can be effectively reduced regardless of the measurement environment.

### BRIEF DESCRIPTION OF THE DRAWINGS

FIG. 1 is a block diagram illustrating an exemplary configuration of semiconductor device in present embodiment.

FIG. 2 is a block diagram illustrating an exemplary configuration of the semiconductor device according to first embodiment

FIG. 3 is a block diagram illustrating an exemplary configuration of the semiconductor device according to second embodiment.

FIG. 4 is a block diagram illustrating an exemplary configuration of the semiconductor device according to third embodiment.

FIG. 5 is a block diagram illustrating an exemplary configuration of the semiconductor device according to fourth embodiment.

FIG. 6 is a block diagram illustrating an exemplary configuration of the biological signal sensor system according to modified embodiment.

### DETAILED DESCRIPTION

Hereinafter, semiconductor device, the biological signal sensor, and the biological signal sensor system according to one embodiment will be described in detail by referring to the drawings. In the specification and the drawings, the same or corresponding form elements are denoted by the same reference numerals, and a repetitive description thereof is omitted. In the drawings, for convenience of description, the calibration may be omitted or simplified. Also, at least some of the embodiments may be arbitrarily combined with each other.

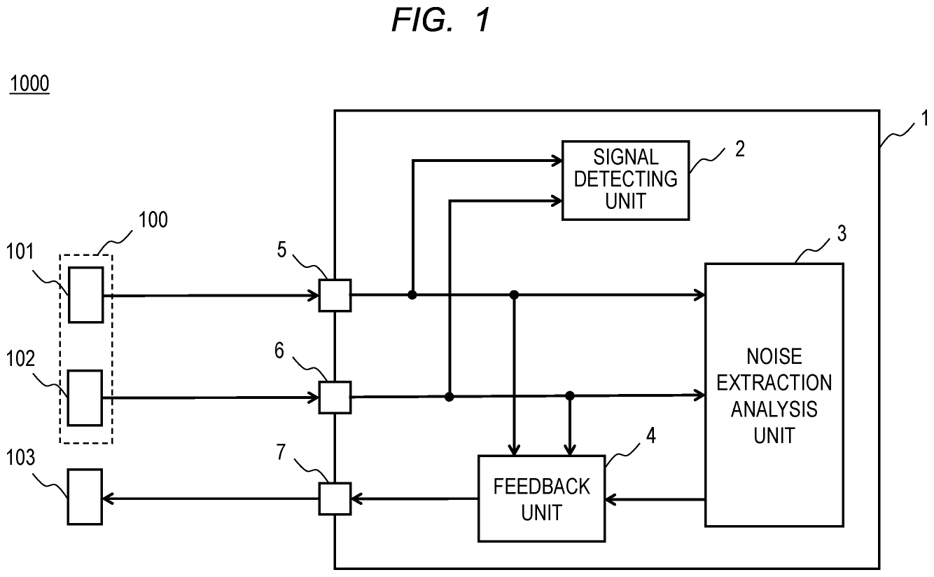

Before describing the details of the embodiment, an outline of the embodiment will be first described. FIG. 1 is a block diagram showing an example of a configuration of a biological signal sensor 1000 according to the outline of the embodiment. The biological signal sensor 1000 includes a semiconductor device 1. The semiconductor device 1 includes a signal detecting unit 2, a noise extraction analysis unit 3, a feedback unit 4, a first terminal 5, a second terminal 6, and a third terminal 7.

The signal detecting unit 2 receives biological signals detected by a pair of signal detection electrodes 100 (a first signal detection electrode 101 and a second signal detection electrode 102) attached to the human body via the first terminal 5 and the second terminal 6, and differentially amplifies the biological signals. The differentially amplified signal is transferred to a personal computer, a portable device, or the like (not shown) and displayed.

The noise extraction analysis unit 3 extracts a signal of a specific frequency component which corresponds to noise from the biological signals received via the first terminal 5 and the second terminal 6. In addition, the noise extraction analysis unit 3 acquires the magnitude of the extracted signal of the specific frequency component.

The feedback unit 4 generates a feedback signal to cancel the signal of the specific frequency component corresponding to noise superimposed on the biological signals received through the first terminal 5 and the second terminal 6. The feedback signal is a signal obtained by inverting and amplifying the biological signals received through the first terminal 5 and the second terminal 6 in accordance with the magnitude of the signal of the specific frequency component extracted by the noise extraction analysis unit 3. The generated feedback signal is output to the feedback electrode 103 (third signal electrode) attached to the human body through the third terminal 7. The common mode noise superimposed on the biological signals detected by the signal detection electrode 100 is reduced by the influence of the signal input from the feedback electrode.

Thus, according to present embodiment, by generating a feedback signal based on the magnitude of the common mode noise (e.g., hum noise), it is possible to generate an appropriate feedback signal depending on the measurement environment. As a result, a biological signal from which common mode noise has been reduced can be obtained, and the biological signal can be accurately monitored.

### First Embodiment

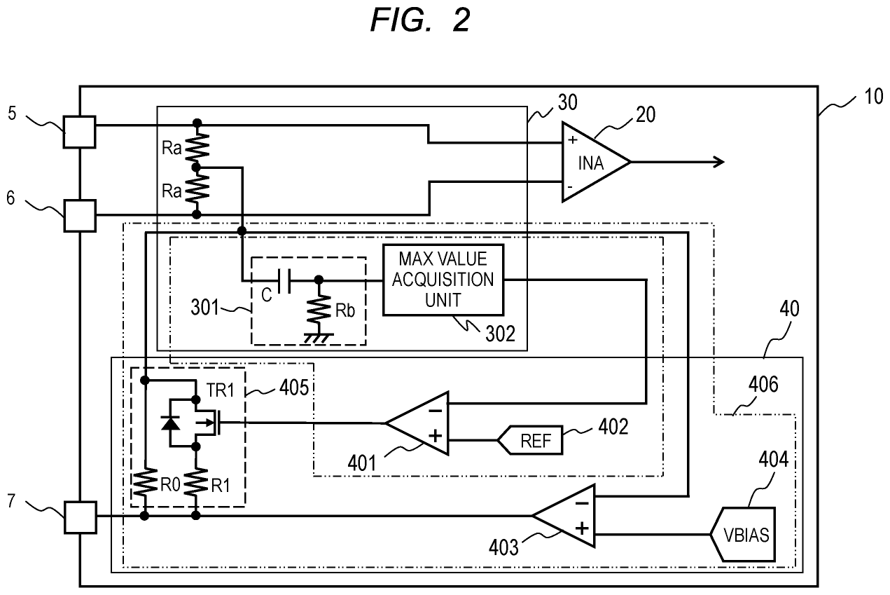

Next, first embodiment will be described. FIG. 2 shows the configurations of semiconductor device 10 according to first embodiment. As shown in FIG. 2, semiconductor device 10 includes a first terminal 5, a second terminal 6, a third terminal 7, a signal detecting unit 20, a noise extraction analysis unit 30, and a feedback unit 40. It should be noted that hum noise, which is one of the causes of common mode noise, is explained as a common-mode noise.

The first terminal 5 and the second terminal 6 are respectively coupled to a pair of signal detection electrodes (not shown) attached to the human body, and receive the first and second biological signals detected by the signal detection electrodes.

The signal detecting unit 20 differentially amplifies the first and second biological signals received via the first terminal 5 and the second terminal 6. For example, an instrumentation amplifier is used as the signal detecting unit 20. The signal differentially amplified by the signal detecting section 20 is transferred to an external device (not shown), and the waveforms of the biological signal based on the transferred signal are displayed or recorded.

The noise extraction analysis unit 30 includes two resistors Ra, a high pass filter 301, and a maximum value acquisition unit 302, extracts signals of specific frequency components superimposed on the first and second biological signals, and outputs the maximum values of the signals of specific frequency components.

The two resistors Ra are coupled in series between the first terminal 5 and the second terminal 6. The intermediate potential signal of the first and second biological signals is output from a connection point of the two resistive elements Ra. The hum noise is superimposed on this intermediate potential signal.

The high pass filter 301 includes a capacitor C and a resistor Rb. That is, the high pass filter 301 uses the intermediate potential signal of the first and second biological signals as an input signal, and extracts the signal of the specific frequency component having a frequency equal to or higher than a predetermined frequency from the intermediate potential signal. In the high pass filter 301, for example, as to extract a signal above the frequency of the commercial power supply as a hum noise (e.g., 50 Hz), the resistance value of the resistor Rb and the capacitance value of the capacitor C are set.

The maximum value acquisition unit 302 acquires and outputs the maximum value of the signal of specific frequency component based on the output of the high pass filter 301. The maximum value acquisition unit 302 includes, for example, an A/D converter (not shown) and a storage unit. The signal of specific frequency component extracted by the high pass filter 301 is sampled and A/D converted. The A/D converted value may be stored in the storage unit. If the A/D converted value of next sampled value is larger than the value stored in the storage unit, the value stored in the storage unit may be updated. That is, the magnitude of the signal of the specific frequency component can be extracted by the maximum value acquisition unit 302.

The feedback unit 40 includes a comparator 401, an operational amplifier 403, and a feedback resistor unit 405.

The comparator 401 compares the maximum value of the signal of the specific frequency component extracted by the noise extraction analysis unit 30 with a predetermined reference value, and outputs the comparison result to the feedback resistor unit 405. When the maximum value of the signal of the specific frequency component is equal to or greater than the reference value, the comparison result becomes an inactive level, when the maximum value of the signal of the specific frequency component is smaller than the reference value, the comparison result becomes an active level. The reference value can be set by digital data, and may be reset via a general-purpose IC after product shipment.

The operational amplifier 403 has an inverting input terminal to which the intermediate potential signal of the first and second biological signals is supplied and a non-inverting input terminal to which the bias voltage 404 is supplied. The biasing voltage is, for example, 1.0 V.

The feedback resistor unit 405 includes resistors R0 and R1 and the transistor TR1 is coupled between the output terminal and the inverting input terminal input terminal of the operational amplifier 403. The resistor R1 and the transistor TR1 are connected in series, the resistor R1 and the transistor TR1 connected in series are coupled in parallel with the resistor R0. The transistor TR1 functions as a switch that turns on when the comparison result of comparator 401 indicates the active level and turns off when the comparison result of comparator 401 indicates an inactive level. By the transistor TR1 is turned on or off, the resistance value between the output terminal of the operational amplifier 403 and the inverting input terminal are varied to change the amplification factor in the operational amplification. In other words, the feedback resistor unit 405, in accordance with the comparison result between the maximum value of the signal of the specific frequency component and the reference value, functions as a variable resistor for changing the amplification factor in the operational amplification.

The output of the operational amplifier 403 is coupled to the third terminal. Focusing on the connection relationship of operational amplifier 403, bias voltage 404, and feedback resistor 405, these components constitute an inverting amplifier 406. Further, since the feedback resistor unit 405 functions as a variable resistance, the inverting amplifier 406 can be said to be a variable gain inverting amplifier. The inverting amplifier inverts and amplifies the intermediate potential signal of the first and second biological signals on which the signal of the specific frequency component is superimposed, generates a feedback signal that cancels the signal of the specific frequency component superimposed on the biological signal, and outputs the feedback signal to the third terminal 7.

Next, the operation of the semiconductor device 10 will be described when the high pass filter 301 is configured to extract a signal of a specific frequency component of the frequency 50 Hz or more of the commercial power supply that becomes hum noise.

The semiconductor device 10 receives the first and second biological signals detected by the pair of signal detection electrodes 100 attached to the human body via the first terminal and the second terminal 6. The signal detecting unit 20 differentially amplifies the first and second biological signals. Since signals corresponding to hum noise superimposed on the first and second biological signals are superimposed on the differential amplified signals, noise appears also in biological signal waveforms displayed by an external device (not shown).

In the noise extraction analysis unit 30, the intermediate potential signal of the first and second biological signals is generated by the two resistors Ra connected in series between the first terminal 5 and the second terminal 6. The signals corresponding to the hum noise is superimposed on the intermediate potential signal. The signal having the frequency corresponding the hum noise (e.g., 50 Hz) or more signals are extracted from the intermediate potential signal by the high pass filter 301. For example, a signal with a frequency component lower than the frequency of the hum noise is important for diagnosing the electrocardiogram, and a signal with a frequency higher than the frequency of the hum noise can be regarded as noise. Therefore, extracting a signal having the frequency corresponding the hum noise (e.g., 50 Hz) or more by the high pass filter 301 is equivalent to extracting a common mode noise signal superimposed on a biological signal. Hereinafter, signal with frequency above the frequency corresponding the hum noise extracted by the high pass filter 301 is referred to as a hum noise signal.

The maximum value of the hum noise signal is compared by a comparator 401 to a predetermined reference value. When the maximum value of the hum noise signal is equal to or greater than the reference value, the comparison result is at the inactive level and the transistor TR1 is turned off. On the other hand, when the maximum value of the noise signal is smaller than the reference value, the comparison result becomes the active level, the transistor TR1 is turned on. The resistance value between the output terminal and the inverting input terminal of the operational amplifier 403 is greater when the maximum value of the hum noise signal is more than the reference value than when smaller than the reference value. Therefore, the amplification factor in the operational amplification increases when the maximum value of the hum noise signal is equal to or greater than the reference value.

The operational amplifier 403 inverts and amplifies the intermediate potential signal with the amplification factor determined as described above. Thus, the inverted amplified signal of the hum noise signal is output to the third terminal 7. The output of the operational amplifier 403 is supplied as a feedback signal to the feedback electrode attached to the human body through the third terminal 7. The first and second biological signals are influenced by the feedback signal and become biological signals from which hum noise has been reduced.

In this manner, by generating a feedback signal reflecting the magnitude of the hum noise signal in the measurement environment and feeding the feedback signal back to the human body, the hum noise can be effectively reduced from the biological signal. That is, since the feedback signal is generated based on the magnitude of the hum noise in the environment at the time of measurement, the hum noise can be appropriately reduced from the biological signal in any measurement environment, and the purpose biological signal can be appropriately displayed or recorded.

### Second Embodiment

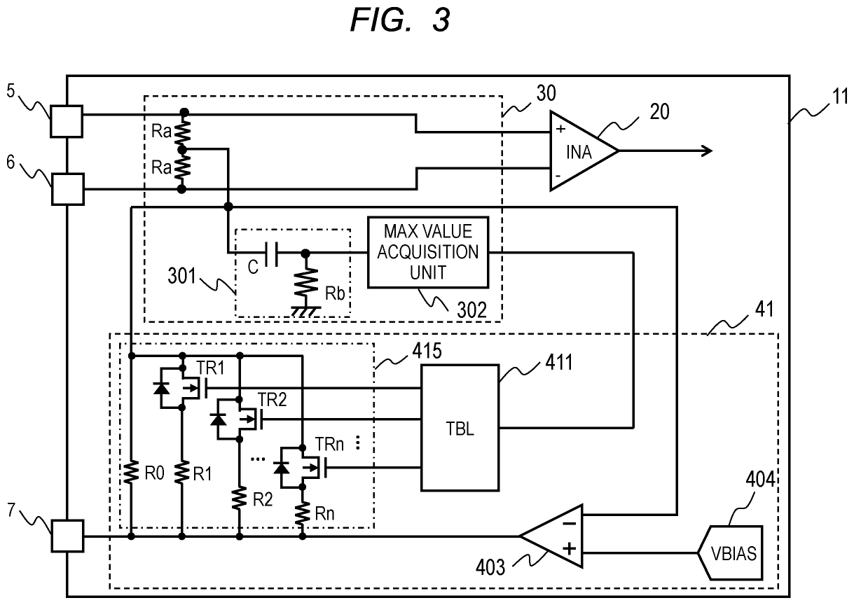

Next, second embodiment will be described. In second embodiment, a feedback unit 41, which is another form of the feedback unit 40 according to first embodiment, will be described. FIG. 3 shows an exemplary configuration of second embodiment feedback unit 41. In second embodiment, the configurations other than the feedback unit 41 of semiconductor device 11 may be similar to those shown in FIG. 2. Therefore, their descriptions are omitted here. In second embodiment, components having the same functions as those in FIG. 2 of the embodiment are denoted by the same reference numerals, and descriptions thereof are omitted.

As shown in FIG. 3, the feedback unit 41 has a conversion table TBL 411 in place of the comparison unit 401 according to first embodiment, and a feedback resistor unit 415 in place of first embodiment according to feedback resistor unit 405. Configurations other than the conversion table TBL 411 and the feedback resistor unit 415 may be the same as those shown in FIG. 2, and therefore, the same reference numerals are given here, and descriptions thereof are omitted.

The conversion table 411 converts the maximum value of the signal of the specific frequency component extracted by the noise extraction analysis unit 30 into a control signal for controlling the feedback resistor unit 415. The conversion table 411 may be set via a general-purpose a general purpose input/output unit.

The feedback resistor 415 includes a resistor R0, a resistor R1 to Rn, and a transistor TR1 to TRn, where n is an integer. A configuration in which the resistors R1 to Rn and the corresponding transistors TR1 to TRn are connected in series is connected in parallel to the resistor R0. Each of transistors TR1 to TRn receives corresponding control signals and turns on or off. By the transistors TR1 to TRn are turned on or off, respectively, the resistance value between the output terminal of operational amplifiers 403 and the inverting input terminal are changed, and the amplification factor in operational amplifiers is varied.

According to present embodiment, as compared with first embodiment, it is possible to finely set the resistance value in the feedback resistor unit 415. Therefore, the amplification factor in the operational amplification can be finely set. Therefore, depending on the measurement environment, it is possible to generate a more appropriate feedback signal. As a result, the hum noise can be appropriately reduced from the biological signal regardless of the measurement environment, and the target biological signal can be appropriately displayed or recorded.

### Third Embodiment

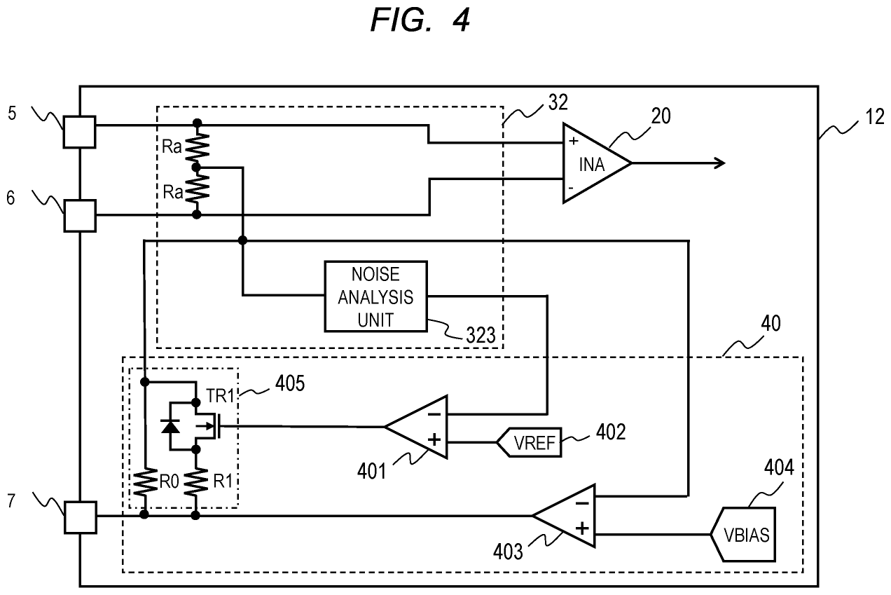

In third embodiment, a noise extraction analysis unit 32, which is another form of the noise extraction analysis unit 30 according to first embodiment, will be described. FIG. 4 shows an exemplary configuration of the noise extraction analysis unit according to third embodiment. In third embodiment, the configurations other than the noise extraction analysis unit 32 included in semiconductor device 12 may be the same as the configurations of semiconductor device 10 shown in FIG. 2. Therefore, components having the same functions as those in FIG. 2 of first embodiment are denoted by the same reference numerals, and description thereof are omitted here.

As shown in FIG. 4, the noise extraction analysis unit 32 includes a noise analysis unit 323. The noise analysis unit 323 performs frequency analysis of the intermediate potential signal of the first and second biological signals. As a result of the frequency analysis by the noise analysis unit 323, the frequency and the amplitude of the signal included in the intermediate potential signal of the first and second biological signals are detected. Therefore, the amplitude of the signal of the specific frequency included in the intermediate potential signal can be extracted by the noise analysis unit 323. For example, the noise analysis 323 extracts the amplitude of the signal having a frequency of 50 Hz of the commercial power supply to be hum noise.

The amplitude of the signal of the specific frequency extracted by the noise analysis unit 323 is compared by a comparator 401 with a reference value. The comparison result of the comparator 401 is supplied to the feedback resistor unit 405. The feedback resistor unit 405 varies, as described above, the resistance value between the output and the inverting input terminal of the operational amplifier 403 in accordance with the comparison result. Thus, the amplification factor in the operational amplifier is varied. The operational amplifier 403 inverts and amplifies the biological signal on which the hum noise is superimposed with the amplification factor, and outputs it as a feedback signal.

Thus, by performing a frequency analysis by the noise analysis unit 323, in accordance with the amplitude of the signal of a specific frequency, the amplification factor in the operational amplification is determined. If the frequency of the signal is known, such as hum noise, the frequency can be identified, and a feedback signal can be generated based on the magnitude of the signal of the specified frequency component. Since the influence of the hum noise is large among the common mode noise superimposed on the biological signal, the hum noise superimposed on the biological signal can be effectively reduced by generating the feedback signal based on the magnitude of the hum noise.

### Fourth Embodiment

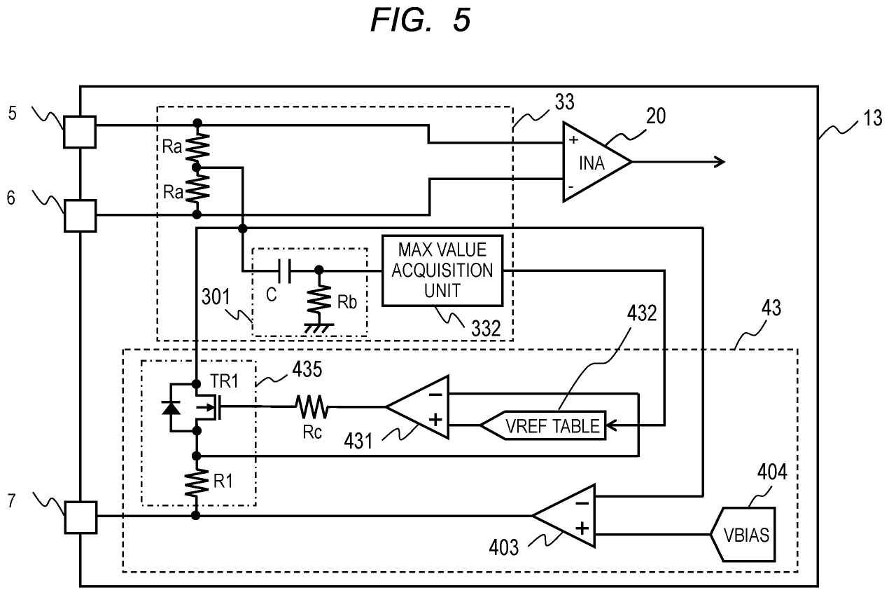

Next, fourth embodiment will be described. In fourth embodiment, a semiconductor device 13, which is another form of the semiconductor device 10 according to first embodiment, will be described. FIG. 5 shows an example of the configurations of the noise analysis extracting unit 33 and the feedback unit 43 included in the semiconductor device 13 according to fourth embodiment. In fourth embodiment, the configurations other than the noise analysis extracting unit 33 and the feedback unit 43 included in semiconductor device 13 may be the same as the configurations of the semiconductor device 10 shown in FIG. 2. Therefore, components having the same functions as those in FIG. 2 are denoted by the same reference numerals, and description thereof are omitted here.

First, the noise extraction analysis unit 33 will be described. The noise extraction analysis unit 33 shown in FIG. 5 includes a maximum value acquisition unit 332 instead of the maximum value acquisition unit 302 among the components included in the noise extraction analysis unit 30 shown in FIG. 2. The maximum value acquisition unit 332 outputs the maximum value of the signal of the specific frequency component of the intermediate potential signal of the first and second biological signal obtained by the high pass filter 301 in a digital value.

The feedback unit 43, as shown in FIG. 5, includes an operational amplifier 431, a reference value table 432, an operational amplifier 403, a bias voltage supply unit 404 and a feedback resistor unit 435.

The reference value table 432 will be described. The reference value table 432 is a correspondence table between the reference value and the maximum value of the signal of the specific frequency component output from the noise extraction analysis unit 30. The reference value corresponding to the maximum value of the signal of the specific frequency component output from the maximum value acquisition unit 332 is selected. The reference value table 432 performs digital to analog conversion of the selected reference value to generate reference voltage, and supplies the reference voltage to the non-inverting input terminal of the operational amplifier 431.

Here, focusing on the relationship the connection between the operational amplifier 431, the transistor TR1 and the resistor R1, the operational amplifier 431, the transistor TR1 and the resistor R1 constitute a current output amplifier. In response to reference voltage supplied to the current output amplifier, the current flowing through the resistor R1 is determined. That is, operational amplifiers 431 control the gate electrodes of the transistor TR1 in response to reference voltage. That is, the on-resistance of the transistor TR1 is controlled.

Thus, the resistance values between the output terminal and the inverting input terminal of the operational amplifier 403 is set based on the on-resistance of the transistor TR1 controlled by the operational amplifier 431, and the resistance R1. The operational amplifier 403 inverts and amplifies the intermediate potential signal of the first and second biological signals with an amplification factor determined based on the resistance values between the output terminal and the inverting input terminal of the operational amplifier 403. The output of operational amplifier 403 is connected to a feedback electrode attached to the human body via third terminal 7, similar to other embodiments. The first and second biological signals are influenced by the feedback signal to become a biological signal from which hum noise has been reduced.

Thus, by selecting reference voltage according to the magnitude of the signal of the specific frequency component corresponding to hum noise, and controlling the on-resistance of the transistor at the current output amplifier, it is also possible to set the amplification factor of the inverting amplifier. Therefore, even if the magnitude of the hum noise changes in accordance with the measurement environment, the feedback signal following the change can be generated, and as a result, the hum noise superimposed on the biological signal can be effectively reduced.

### Modified Example

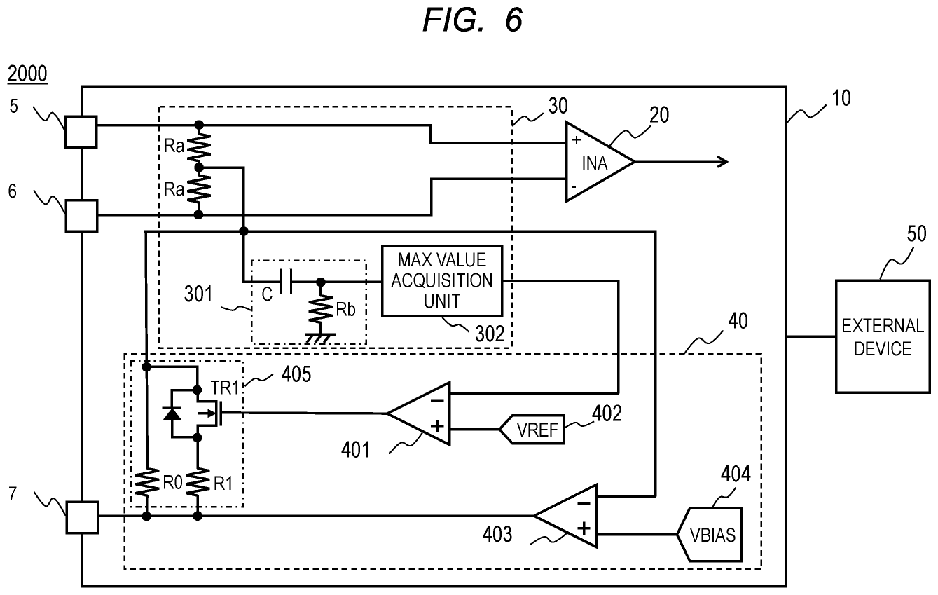

FIG. 6 shows modified example. FIG. 6 is a biological sensor system comprising a semiconductor device 10 and an external device 50. The external device 50 is, for example, a personal computer or a portable device to display and analyze biological signals and the like transferred from semiconductor device 10.

The external device 50, not only the output of the signal detecting unit 2 of semiconductor device 10, the output of the comparator 401 or the maximum value acquisition unit 302 is transferred. The external device 50 receives the outputs of the signal detecting unit 2 and displays the biological signal waveforms. Further, the external device 50 displays the hum noise occurrence status in the measuring environment based on the output of the comparator 401 or the maximum value acquisition unit 302 which varies according to the magnitude of the noise superimposed on the biological signal. The user of the biological signal sensor can change the measurement environment in accordance with the biological signal waveforms displayed on the external device 50 and the hum noise occurrence state in the measurement environment. As a result, a more accurate biological signal waveform can be obtained.

Although the invention made by the inventor has been specifically described based on the embodiment, the present invention is not limited to the embodiment already described, and it is needless to say that various modifications can be made within a range not deviating from the gist thereof. For example, in the feedback unit, the signal of the specific frequency component extracted by the noise extraction analysis unit may be compared with the reference value. The reference value to be compared in the comparator may be reset after product shipment using general-purpose Input/Output ports.

What is claimed is:

1. A semiconductor device, comprising:
a first terminal receiving a first signal;
a second terminal receiving a second signal;
a noise extraction analysis unit coupled to the first and the second terminal to extract a signal of a specific frequency component from the first and the second signal;
a feedback unit configured to generate a feedback signal based on a magnitude of the signal of the specific frequency component to cancel the signal of the specific frequency component superimposed on the first and the second signal, and
a third terminal outputting the feedback signal to outside.

2. The semiconductor device according to claim 1,
wherein the feedback unit comprises an inverting amplifier inverting and amplifying an intermediate potential signal of the first and the second signal with an amplification factor set based on the magnitude of the signal of the specific frequency component, and
wherein the feedback signal is an output signal of the inverting amplifier.

3. The semiconductor device according to claim 2,
wherein the feedback unit further comprises a comparator comparing the magnitude of the signal of the specific frequency component with a reference value,
wherein the inverting amplifier comprises:
an operational amplifier having an inverting input terminal and an output terminal, and
a feedback resistor unit coupled between the inverting input terminal and the output terminal and having a resistance value changed by a comparison result of the comparator.

4. The semiconductor device according to claim 1,
wherein the noise extraction analysis unit includes a filter extracting a signal of the specific frequency component of an intermediate potential signal of the first and the second signal.

5. The semiconductor device according to claim 1,
wherein the noise extraction analysis unit comprises a frequency analysis unit performing frequency analysis a signal of the specific frequency component of an intermediate potential signal of the first and the second signal to output an amplitude of the signal of the specific frequency component as a magnitude of the signal of the specific frequency component.

6. The semiconductor device according to claim 3,
wherein the feedback resistor unit comprises a first resistor and a second resistor coupled in series to a switch,
wherein the second resistor and the switch coupled in series are coupled in parallel with the first resistor, and
wherein the switch configured to turn on or off in response to the comparison result.

7. The semiconductor device according to claim 1,
wherein the feedback unit comprises:
an operational amplifier having an inverting input terminal and an output terminal;
a feedback resistor unit coupled between the inverting input terminal and the output terminal;
a reference voltage table configured to select a reference voltage in response to the magnitude of the signal of the specific frequency component; and
a current output amplifier configured to control a current flowing through the feedback resistor unit in response to the selected reference voltage,
wherein the feedback resistor unit includes a resistor and a transistor coupled in series to the resistor,
wherein the current output amplifier controls a gate electrode of the transistor, and
wherein the feedback signal is an output of the operational amplifier.

8. The semiconductor device according to claim 1 further comprising a differential amplifier,
wherein the first signal is a first biological signal detected by a first signal detection electrode attached to human body,
wherein the second signal is a second biological signal detected by a second signal detection electrode attached the human body,
wherein the feedback signal is feedbacked to the human body through a feedback electrode, and
wherein the differential amplifier differentially amplifies the first biological signal and the second biological signal.

9. The semiconductor device according to claim 1,
wherein a frequency of the signal of the specific frequency component is equal to or higher than the commercial power supply frequency.

10. The semiconductor device, comprising:
a first terminal receiving a first biological signal;
a second terminal receiving a second biological signal;
a third terminal outputting a feedback signal;
a noise extraction analysis unit coupled to the first and the second terminal to extract a signal of a specific frequency component from an intermediate potential signal of the first and the second biological signal; and
a variable gain inverting amplifier receiving the intermediate potential signal,
wherein an amplification factor is set based on a magnitude of the signal of the specific frequency component.

11. A biological signal sensor comprising:
a first terminal receiving a first biological signal detected by a first signal detection electrode attached to a human body;
a second terminal receiving a second biological signal detected by a second signal detection electrode attached to the human body;
a differential amplifier differentially amplifying the first biological signal and the second biological signal;
a noise extraction analysis unit configured to extract a signal having a specific frequency component from an intermediate potential signal of the first and the second biological signal;
a feedback unit configured to generate a feedback signal based on a magnitude of the signal of the specific frequency component to cancel the signal of the specific frequency component superimposed on the first and the second biological signal; and
a third terminal coupled to a feedback electrode attached to the human body to output the feedback signal to the feedback electrode.

12. A biological signal sensor system comprising:
the biological signal sensor according to claim 11, and
an external device receiving an output of the differential amplifier in the biological signal sensor and displaying a biological signal waveform.

13. The biological signal sensor system according to claim 12,
wherein the external device displays a noise condition in a measurement environment based on the magnitude of the signal of the specific frequency component which is extracted by the noise extraction analysis unit in the biological signal sensor.
